(19) United States
(12) Patent Application Publication
(10) Pub. No.: US 2016/0296135 A1
(43) Pub. Date: Oct. 13, 2016

(54) ELECTRICAL IMPEDANCE TOMOGRAPHY DEVICE

(71) Applicant: K-HEALTHWEAR CO., LTD., Daejeon (KR)

(72) Inventors: Hoi Jun YOO, Daejeon (KR); Seon Ju HONG, Seoul (KR)

(73) Assignee: K-HEALTHWEAR CO., LTD., Daejeon (KR)

(21) Appl. No.: 15/100,987

(22) PCT Filed: Nov. 19, 2014
(86) PCT No.: PCT/KR2014/011121

§ 371 Date: Jun. 2, 2016

(30) Foreign Application Priority Data
Dec. 4, 2013 (KR) 10-2013-0149797

(51) Int. Cl. A61B 5/053; A61B 5/00

(52) U.S. Cl. CPC A61B 5/0536; A61B 5/0022; A61B 5/742; A61B 5/6804; A61B 5/4312; A61B 5/7225; A61B 2562/046; A61B 2562/164; A61B 2560/0214

(57) ABSTRACT

Disclosed is an electrical impedance photographing patch comprising a plurality of electrodes contacting with skin, applied with an electrical signal and arranged on a flexible substrate, wherein the photographing patch measures impedance, i.e., an electrical signal, of the skin of a measurement target placed between the plurality of electrodes and transmits the electrical signal to an external device which restores the electrical signal into a three-dimensional tomographic image and displays the tomographic image.

### CROSS REFERENCE TO PRIOR APPLICATIONS

This application is a National Stage Application of PCT International Patent Application No. PCT/KR2014/011121 filed on Nov. 19, 2014, under 35 U.S.C. §371, which claims priority to Korean Patent Application No. 10-2013-0149797 filed on Dec. 4, 2013, which are all hereby incorporated by reference in their entirety.

### BACKGROUND

The present invention relates to an electrical impedance tomography device, and more specifically, to an electrical impedance tomography device, which can detect electrical characteristics of the inner parts of a measurement target so that a general user may diagnose existence of a disease by himself or herself in an early stage.

As a method of diagnosing a lesion inside a body, a method of measuring physical properties or physiological attributes of the inner structure of a human body from the outside of the human body and determining a normal state or an abnormal state based on the measurement is chiefly used.

Particularly, it is reported that electrical impedance of a cancer part has a value smaller than that of the impedance of a normal part.

Generally, in order to take an image of the inner structure of a human body or an object, density of the body or distribution of temperature in the body is measured using X-ray, MRI, ultrasound, heat or the like.

Recently, an Electrical Impedance Tomography (EIT) technique actively studied from the late 1970s is used as a new method of imaging.

The electrical impedance tomography is a technique of imaging the resistivity of an inner part of a human body by attaching a plurality of electrodes on the surface of the human body, applying current through some of the electrodes, and then measuring voltage through other electrodes attached on the surface of the human body.

Since different parts of a human body have different electrical impedance values, the imaging can be accomplished by using the resistivity of the inner parts of the human body as described above.

However, since there is no scientific and objective early self-diagnostic equipment in the prior art, only a very inaccurate measurement method has been particularly used for the convenience of early self-diagnosis of breast cancer at home, and there is a problem in that an X-ray mammography diagnosis should be taken at a special clinic for accurate diagnosis.

Conventionally, when breast cancer is diagnosed using an electrical impedance tomography device in Dartmouth College of USA, a patient lies face down on a bed having holes and puts the breasts into semi-spherical containers through the holes to contact the breasts with electrodes through a conductive liquid filled in the semi-spherical containers. However, this method cannot be used since noises in the images are too severe. In addition, although the TCI Company of USA used a detector having dozens of electrode rods tied together after attaching the detector to the breasts of a patient lying on a bed, it is difficult to use the detector at home due to its volume and weight. Although the impedance breast cancer diagnostic device of a two-dimensional image sold by Siemens in 1999 does not have a method of measuring distribution of impedance using a small scanner while a patient is lying, it is also an expert device which can be used only by an expert. Since the product of the ZTech company of USA arranges electrodes by way of restricting the number of electrodes, a real image is difficult to be implemented, and as a result, this is an expert device which displays only the differences in the features of the left and right breasts. As described above, the conventional techniques have a problem in that since the devices are too big or the images are two-dimensional and, in addition, the devices are installed only in a clinic and can be read only by an expert, they cannot be distributed widely for household purposes. Accordingly, in order to solve such a problem, the present invention provides a photographing patch of a modular form, which includes, in one piece, an electrode layer for three-dimensionally and easily attaching a plurality of micro-electrodes to fit to the shape of a body, a wire layer configuring a wire for transmitting and receiving signals through the electrodes, and a circuit layer provided with a semiconductor integrated circuit for creating and processing three-dimensional images and transmitting and receiving the three-dimensional images to and from an external device.

### PRIOR ART

(Patent Document 1) Korean Patent Publication No. 10-2005-0013407

### SUMMARY

Therefore, the present invention has been made in view of the above problems, and it is an object of the present invention to provide an electrical impedance tomography device, which can stably connect a large number of soft electrodes without reluctance by directly attaching the electrodes on the surface of the skin, manufacture the whole device in the form of a general brassiere by integrating all electronic functions in a semiconductor chip, and collect, image-process and analyze data by simply handling the device through an application of a smart phone or a tablet computer.

To accomplish the above object, according to one aspect of the present invention, there is provided a photographing patch including a plurality of electrodes contacting with the skin, applied with an electrical signal and arranged on a flexible substrate, in which the photographing patch measures impedance, i.e., an electrical signal, of the skin of a measurement target placed between the plurality of electrodes and transmits the electrical signal to an external device which restores the electrical signal into a three-dimensional tomographic image and displays the tomographic image.

An electrode plate, a wire plate and a circuit plate configuring the substrate may be formed of any one or more of a paper product, a natural fiber, a synthetic fiber, non-woven cloth, polymer, plastic, pulp, paper and silicon rubber.

A cover configuring the substrate is formed of a flexible material or plastic.

The electrodes, wires and circuits configuring the substrate may be formed by printing an ink containing a conductive material, adhering cloth, non-woven cloth, paper or a flexible substrate having conductivity, infiltrating a conductive organic matter, configuring a metallic sticker or adhering a small metal plate.

The photographing patch may be a flexible brassiere shape targeting breasts of a woman as a measurement target.

According to another aspect of the present invention, there is provided an electrical impedance tomography device including: a photographing patch including a plurality of electrodes contacting with the skin, applied with an electrical signal and arranged on a flexible substrate, the photographing patch for measuring an electrical signal output through the skin of a measurement target placed between the plurality of electrodes; and an external device for receiving the electrical signal measured through the photographing patch, restoring electrical impedance into a three-dimensional tomographic image, and displaying the tomographic image.

An electrode plate, a wire plate and a circuit plate configuring the substrate may be formed of any one or more of a paper product, a natural fiber, a synthetic fiber, non-woven cloth, polymer, plastic, pulp, paper and silicon rubber.

A cover configuring the substrate is formed of a flexible material or plastic.

Circuits configuring electrode, wire and substrate layers may be formed by printing an ink containing a conductive material, adhering cloth, non-woven cloth, paper or a flexible substrate having conductivity, infiltrating a conductive organic matter, configuring a metallic sticker or adhering a small metal plate.

The plurality of electrodes arranged on the substrate may be arranged in a shape of a regular pattern to evenly contact throughout the skin of the management target.

The shape of a regular pattern of the plurality of electrodes may be a radial or matrix shape.

The photographing patch may include a control chip as a module of one piece, and the control chip controls the device to select some of the plurality of electrodes to supply current and select other electrodes to measure voltage, converts an analog signal measured by the plurality of electrodes into a digital signal, and transmits the digital signal to the external device.

The photographing patch may include: an electrode layer provided with the plurality of electrodes; a wire layer stacked on the top of the electrode layer and provided with a wire electrically connected to the electrodes; and a circuit layer stacked on the top of the wire layer and provided with an electrode connection unit electrically connected to the wire.

The electrode layer may include: an electrode plate of a plate shape; and an electrode terminal configured of a plurality of electrodes and provided in a radial shape from the center of the electrode plate.

The electrode terminal may be configured of a predetermined number of electrodes and configured in plurality to be equally spaced in the circumferential direction.

The wire layer may include: a wire plate of a plate shape; a connection hole formed to penetrate the wire plate at a position corresponding to the electrode; and a wire formed to be extended from the connection hole toward the center of the wire plate.

The circuit layer may include: a circuit plate of a plate shape; an electrode connection unit electrically connected to the wire and provided in plurality on the circuit plate to be equally spaced in the circumferential direction; and a control chip for supplying an electrical signal to the electrode, receiving an electrical signal output from the electrode and transferring the electrical signal to the external device.

The control chip may include: a switching unit for turning on or off electrical connection to the electrode; a current generation unit for generating an electrical signal applied to the electrode; a voltage sensor unit for receiving an electrical signal output from the electrode; a communication unit for transmitting the electrical signal input through the voltage sensor unit to the external device; and a control unit for controlling the switching unit to select an electrode for receiving the electrical signal generated through the current generation unit and electrically connect electrodes other than the electrode receiving the electrical signal to the voltage sensor unit.

A communication connection unit connected to the communication cable may be provided in the circuit layer.

A cover stacked to cover the electrode connection unit may be provided on the top of the circuit layer.

The electrode layer and the wire layer may be electrically connected to each other by inserting a conductive material in a connection hole formed in the wire layer; the wire layer and the circuit layer may be electrically connected to each other by inserting an electrode bar inserted in the connection hole formed near the center portion of the wire layer and protruded upward into the electrode connection unit of the circuit layer to penetrate the circuit layer; and the circuit layer and a cover may be connected to each other by forming an insertion hole concave upward on the bottom of the cover and forcibly inserting the electrode bar penetrating the circuit layer into the insertion hole.

The wire layer and the circuit layer may be electrically connected to each other by inserting an electrode bar inserted in the electrode connection unit of the circuit layer and protruded downward into a connection hole of the wire layer.

Impedance may be measured at a ratio of current to voltage by grouping the plurality of electrodes in pairs of two, applying the current to a certain pair of electrodes, and measuring the voltage between the electrodes of the other electrode pairs.

The electrical impedance tomography device may further include a reference patch configured to include a reference electrode contacting with the skin of a position spaced apart from the photographing patch to measure impedance between the photographing patch attached at a position of the measurement target and the reference patch.

The reference patch may be attached to a cover of the external device or a bar shape object that can be held with a palm.

The reference patch may further include a photographing button.

The reference electrode may be a pair, and impedance may be measured at a ratio of current to voltage by applying the current between any one of the reference electrodes and any one of the electrodes provided in the photographing patch and measuring the voltage between the other reference electrode and the other electrodes provided in the photographing patch.

After calculating an impedance matrix, the external device may calculate distribution of impedance inside the skin by tracing back the internal structure of the skin using the impedance matrix and three-dimensionally display an impedance tomographic image according to depth of the skin using the distribution of impedance.

The external device may direct a control chip embedded in the photographing patch to control the photographing patch to measure surface contact impedance of a part of a body to be measured, determine whether or not an image can be taken based on the surface contact impedance, and display the image.

Since an interface for wired or wireless communication is formed in the circuit layer configuring the photographing patch, a measured electrical signal can be transmitted to the external device and displayed thereon, and a control signal can be received from the external device.

The photographing patch may be supplied with power from the external device in the case of a wired communication.

The photographing patch may be supplied with power by attaching a small battery to the circuit layer in the case of a wireless communication.

According to the electrical impedance tomography device of the present invention as described above, there is an effect of stably connecting a large number of electrodes without reluctance by directly attaching the soft electrodes on the surface of the skin, manufacturing the whole device in the form of a general brassiere by integrating all electronic functions in a semiconductor chip, and collecting, image-processing and analyzing data by simply handling the device through an application of a smart phone or a tablet computer.

### BRIEF DESCRIPTION OF THE DRAWINGS

FIGS. 1 and 2 are views showing an electrical impedance tomography device according to an embodiment of the present invention.

FIGS. 3 and 4 are views showing an electrode layer configuring an electrical impedance tomography device according to an embodiment of the present invention.

FIG. 5 is a view showing a wire layer configuring an electrical impedance tomography device according to an embodiment of the present invention.

FIG. 6 is a view showing a circuit layer and a cover configuring an electrical impedance tomography device according to an embodiment of the present invention.

FIG. 7 is a block diagram showing a control chip configuring an electrical impedance tomography device according to an embodiment of the present invention.

FIGS. 8 to 12 are views showing the process of manufacturing an electrical impedance tomography device according to an embodiment of the present invention.

FIGS. 13 to 15 are views showing different cases of using an electrical impedance tomography device according to an embodiment of the present invention.

### DESCRIPTION OF SYMBOLS

100: Photographing patch 230: Electrode

900: External device

### DETAILED DESCRIPTION

The preferred embodiments of the present invention will be hereafter described in detail with reference to the accompanying drawings so that those skilled in the art may easily embody the present invention.

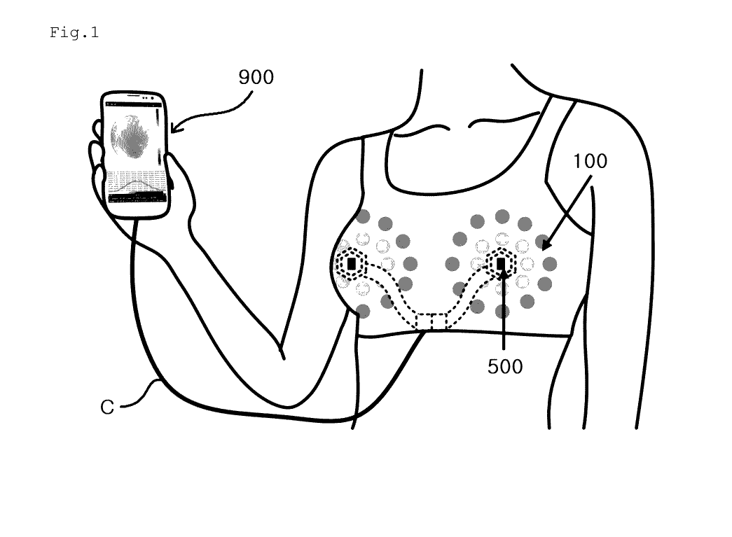

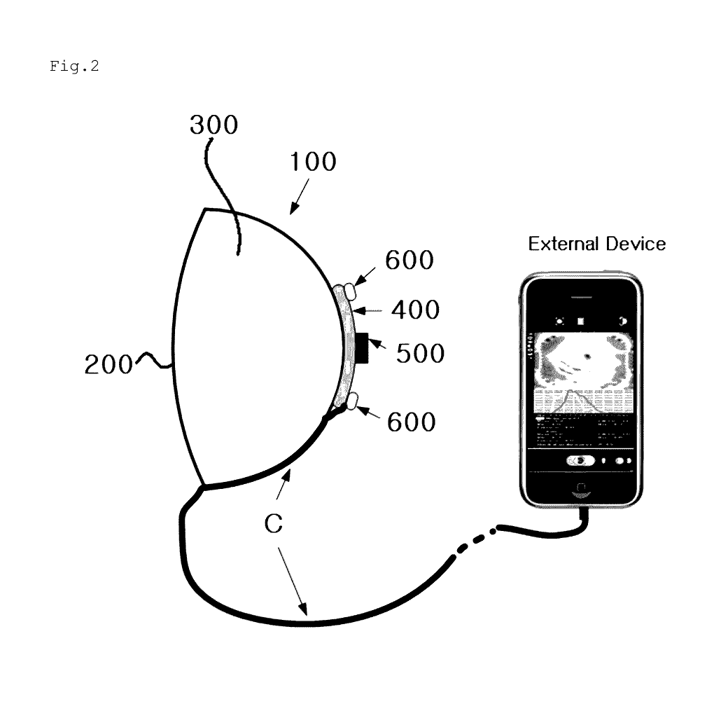

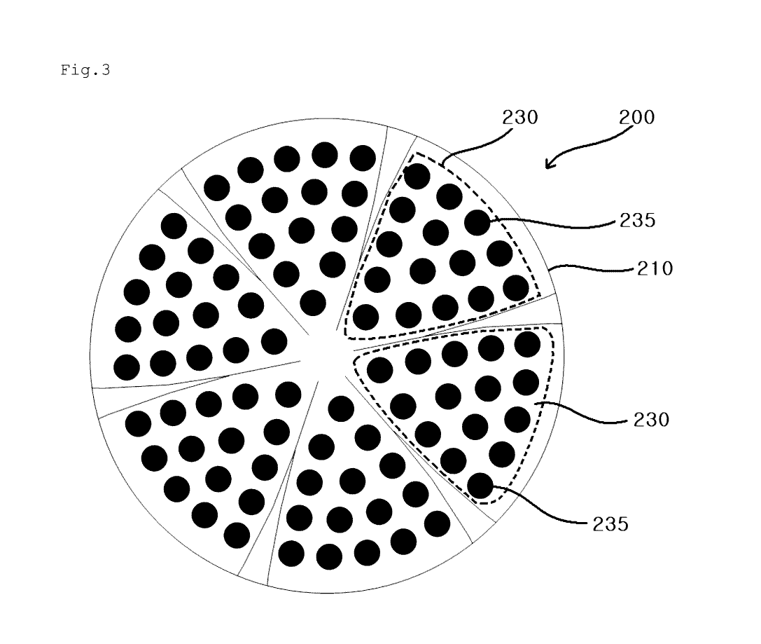

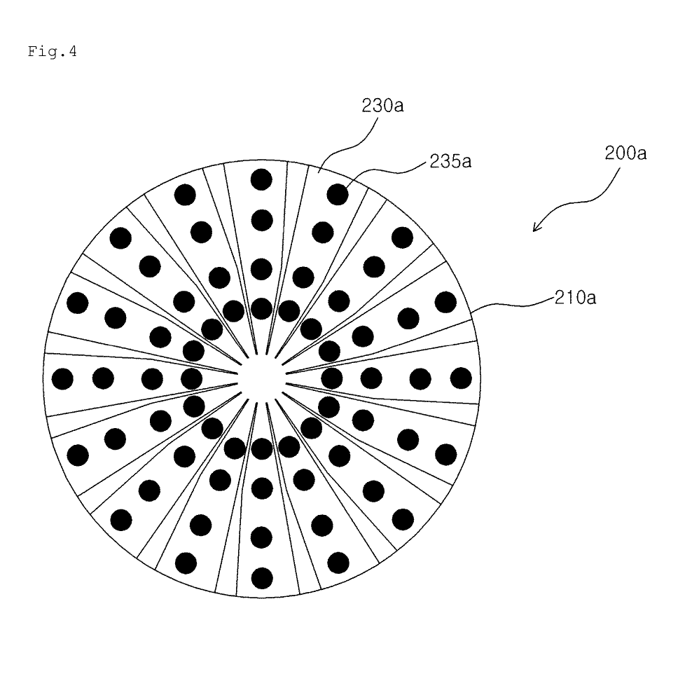

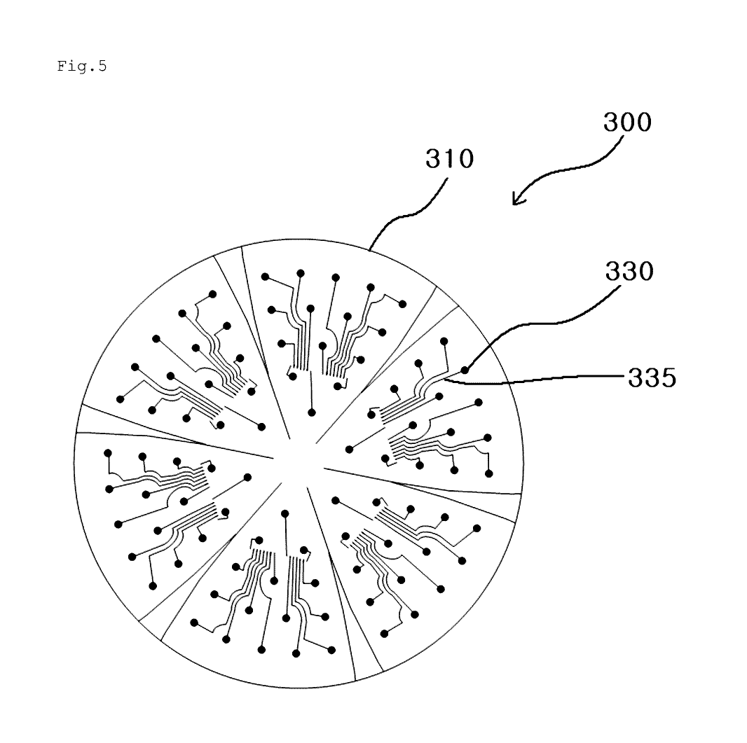

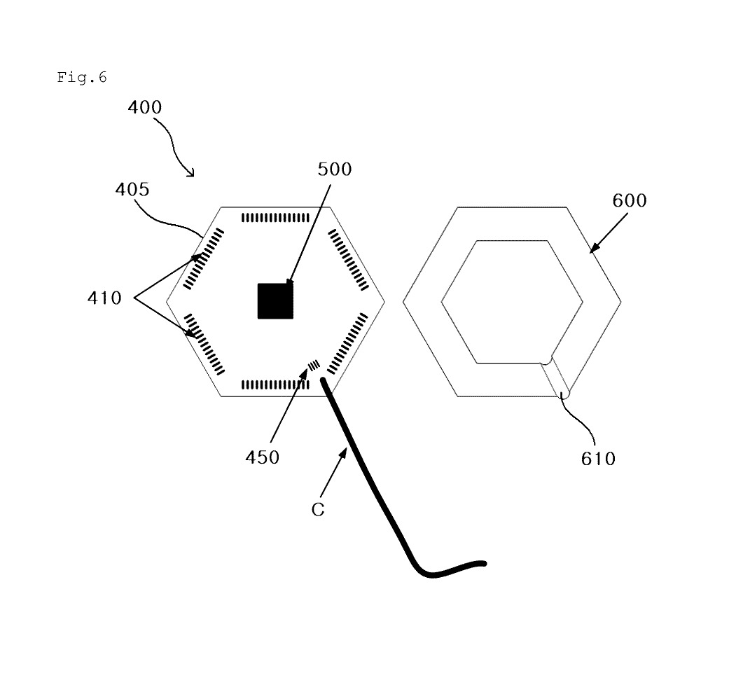

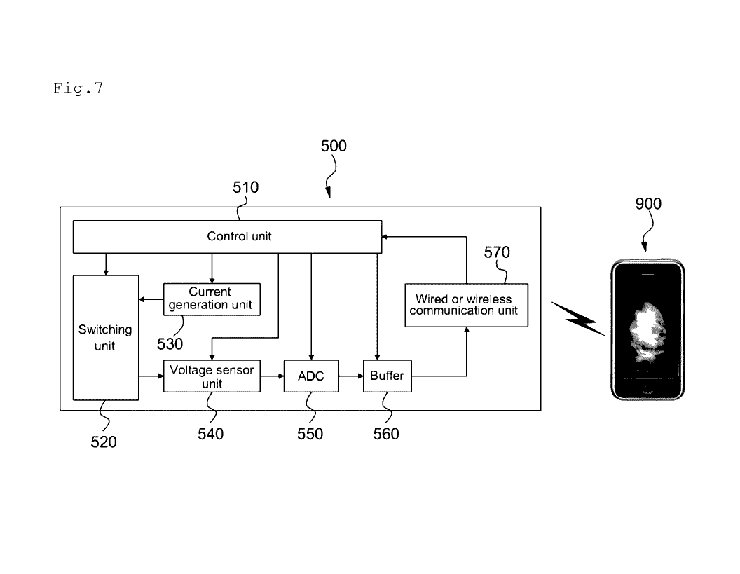

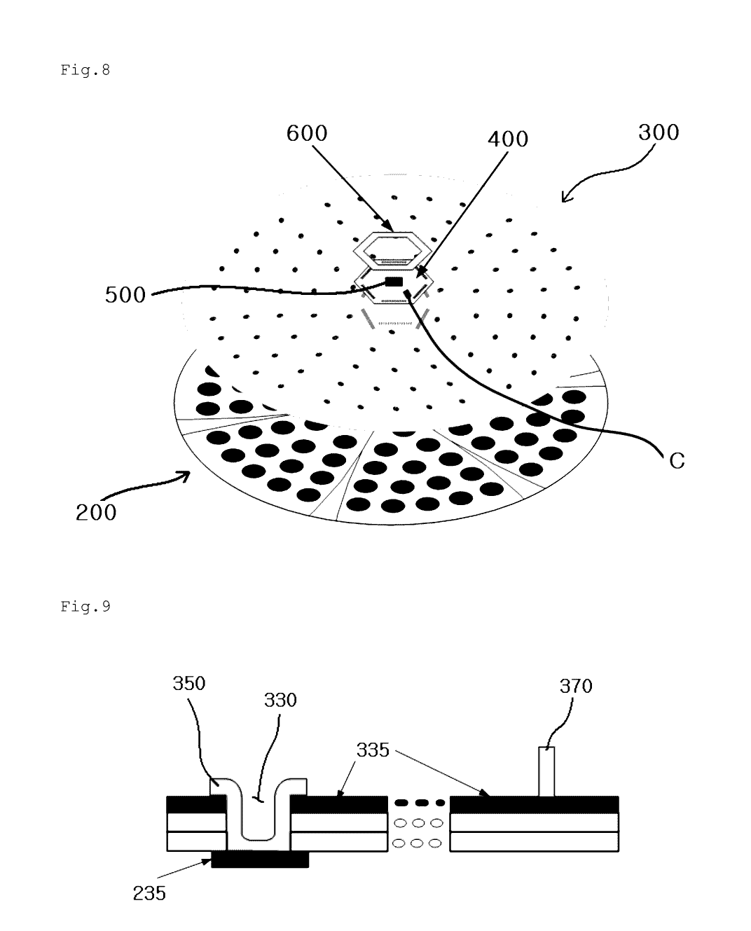

FIGS. 1 and 2 are views showing an electrical impedance tomography device according to an embodiment of the present invention, FIGS. 3 and 4 are views showing an electrode layer configuring an electrical impedance tomography device according to an embodiment of the present invention, FIG. 5 is a view showing a wire layer configuring an electrical impedance tomography device according to an embodiment of the present invention, FIG. 6 is a view showing a circuit layer and a cover configuring an electrical impedance tomography device according to an embodiment of the present invention, FIG. 7 is a block diagram showing a control chip configuring an electrical impedance tomography device according to an embodiment of the present invention, and FIGS. 8 to 12 are views showing the process of manufacturing an electrical impedance tomography device according to an embodiment of the present invention.

As shown in FIG. 1, an electrical impedance tomography device according to an embodiment of the present invention is configured to include a photographing patch 100 and an external device 900 connected to the photographing patch 100 through a communication cable C.

The photographing patch 100 includes a plurality of electrodes evenly arranged in the form of a predetermined pattern such as an radial or matrix shape at equal space to contact with the skin and to be applied with an electrical signal, measures an electrical signal output from the skin placed between the plurality of electrodes through the electrodes, and transmits three-dimensional image source information (relative positions between electrodes, depth of the electrodes, a pattern of arranging the electrodes, distribution of impedance between the electrodes), which is a digital signal converted from the measured analog electrical signal, to the outside, and, as shown in FIG. 2, the photographing patch 100 is configured to include an electrode layer 200, a wire layer 300, a circuit layer 400, a control chip 500 and a cover 600.

As shown in FIGS. 3 and 4, the electrode layer 200 and 200a is configured to include an electrode plate 210 and 210a of a disk shape and an electrode terminal 230 and 230a configured of a plurality of electrodes 235 and 235a provided in a radial shape from the center of the electrode plate 210 and 210a.

The electrode terminal 230 and 230a is configured in plurality to be equally spaced in the circumferential direction.

In the present invention, six electrode terminals 230 respectively configured of fifteen electrodes 235 are provided to be equally spaced in the circumferential direction as shown in FIG. 3, and sixteen electrode terminals 230a respectively configured of four electrodes 235a are provided to be equally spaced in the circumferential direction as shown in FIG. 4.

The electrodes 235a shown in FIG. 4 are formed in the shape of concentric circles having different diameters and arranged to have the same number of electrodes 235a in each of the circles.

Here, although a radial array of equal space is mainly described as the arrangement of the electrodes, it includes evenly arranging the electrodes throughout the skin of a measurement target when the patch contacts with a part of the body and utilizing information on a three-dimensional electrode array pattern such as the distance between the electrodes, depth of the electrodes and the like. That is, it includes arranging a plurality of electrodes as dots which make pixels, in the form of a matrix according to a rule.

The wire layer 300 is stacked on the top of the electrode layer 200, and as shown in FIG. 5, it is configured to include a wire plate 310 of a disk shape the same as that of the electrode plate 210, a connection hole 330 formed to penetrate the wire plate 310 at a position corresponding to the electrode 235, and a wire 335 formed to be extended from the connection hole 330 toward the center of the wire plate 310.

Since the wire 335 is electrically connected to the electrode 235 and is also electrically connected to the control chip 500 described below, the wire layer 300 performs a function of electrically connecting the electrode 235 and the control chip 500.

An electrode bar 370 is inserted into the connection hole 330 formed near the center of the wire layer 300 and protruded upward, and the wire 335 is electrically connected to the circuit layer 400 through the electrode bar 370.

The circuit layer 400 is stacked on the top of the wire layer 300, and as shown in FIG. 6, it is configured to include a circuit plate 405 of a hexagonal plate shape, an electrode connection unit 410 provided in plurality to be equally spaced in the circumferential direction on the circuit plate 405, and the control chip 500 formed at the center portion to supply an electrical signal to the electrode 235, receive an electrical signal output from the electrode 235 and transfer the electrical signal to the external device 900.

Since the electrode bar 370 passing through the wire layer is connected to the electrode connection unit 410, the electrode connection unit 410 is electrically connected to the wire 335.

The control chip 500 is provided in the form of a semiconductor chip, and as shown in FIG. 7, it is configured to include a switching unit 520, a current generation unit 530, a voltage sensor unit 540, a conversion unit 550, a buffer unit 560, a communication unit 570 and a control unit 510.

The switching unit 520 turns on or off the electrical connection between the electrode 235 and the control chip 500 to select an electrode to be applied with an electrical signal and select an electrode which will output an electrical signal.

The current generation unit 530 generates alternating current, which is an electrical signal to be applied to the electrode, and it supplies the current to the electrode 235 selected through the switching unit 520.

The voltage sensor unit 540 measures voltage, which is an electrical signal output from the electrode 235 selected through the switching unit 520.

The conversion unit 550 converts an analog signal of a voltage signal measured by the voltage sensor unit 540 into a digital signal.

The buffer unit 560 temporarily stores the voltage signal converted into the digital signal through the conversion unit 550.

The communication unit 570 receives a control signal from the external device 900 and transmits data to the external device 900, and it wiredly or wirelessly transmits the digital signal stored in the buffer unit 560 to the external device 900.

The control unit 510 receives the control signal transmitted from the external device 900, selects an electrode 235 to be supplied with the current generated by the current generation unit 530, and selects an electrode 235 for measuring output voltage through the voltage sensor unit 540.

In addition, the control chip may include a function for correcting the digital signal and generating a three-dimensional image signal.

Since the control chip 500 of the circuit layer 400 includes the communication unit 570, the circuit layer 400 can be directly connected to the external device 900 through a communication cable C or the like.

A communication connection unit 450 connected to the communication cable C is provided in the circuit layer 400.

Accordingly, although almost one hundred of wires 335 are connected between the circuit layer 400 and the wire layer 300, only a small number of wires conforming to the standard such as USB or the like can be connected between the circuit layer 400 and the external device 900.

Although it is shown in the present invention that a wired communication method such as USB, PCI or the like is used as a method of communicating with the external device 900 through the communication cable C, a wireless communication such as Bluetooth or the like also can be adopted, and when the communication unit 570 is also implemented as a wireless communication unit in the control chip 500, the communication cable C connected to the external device 900 is not needed.

The control chip 500 may be supplied with power from the external device 900 in the case of a wired communication, or the power can be supplied by installing a small battery (not shown) on the top of the circuit layer 400 or separately installing the battery outside the external device in the case of a wireless communication.

The cover 600 is stacked on the top of the circuit layer 400 to cover the electrode connection units 410 for the stability of the portions electrically connecting the circuit layer 400 and the wire layer 300.

As shown in FIG. 6, the cover 600 may be provided in the shape of a hexagonal band having a penetrated center portion.

A cable groove 610 concave upward is formed at a side of the cover 600 corresponding to a position where the communication cable C connected to the communication connection unit 450 passes through to accommodate the communication cable C.

The cable groove 610 can be formed as a hole at a corner of the cover 600 or can be provided in a form cutting a portion of the cover 600.

A soft material such as cloth, non-woven cloth or a flexible substrate can be used as a material of the electrode layer 200, the wire layer 300 and the circuit layer 400 configuring the photographing patch 100 according to the present invention.

In addition, it is possible to selectively use a publicly known material such as woven cloth, non-woven cloth, a mat, a paper or a combination of these manufactured using an inorganic or organic fiber such as polymer, plastic, pulp, paper, silicon rubber, a glass fiber, an aluminum fiber, a polyester fiber, a polyamide fiber or the like or a composite material formed by applying resin varnish thereon, or it is possible to use a material such as a plastic material such as a polyamide-based resin fiber, a polyester-based resin fiber, a polyolefin-based resin fiber, a polyimide-based resin fiber, an ethylene vinyl alcohol polymer material, a polyvinyl alcohol-based resin material, a polyester chloride-based resin material, a polyvinylidene chloride-based resin material, a polystyrene-based resin material, a corona discharge process, a plasma process, an ultraviolet irradiation process, an electron irradiation process, a flame plasma process, an ionic process, various kinds of surface process or the like on the plastic material.

In addition, it is possible to color these materials using a dye.

The conductive portions such as the electrode 235 provided on the electrode layer 200, the wire 335 provided on the wire layer 300 and the like can be manufactured by printing an ink containing an electrically conductive material, cutting and attaching cloth, non-woven cloth or a flexible substrate having electrical conductivity, or applying or infiltrating a conductive organic matter such as a metallic sticker, a PDOT or the like.

In addition, the conductive portions can be manufactured in a method of adhering or inserting and fixing a small metal plate in the flexible substrate.

As shown in FIG. 8, the circuit layer 400 is manufactured in a form having an area smaller than those of the wire layer 300 and the electrode layer 200, in which the circuit plate 405 is formed of cloth, non-woven cloth or a flexible substrate (polymer, plastic, pulp, paper, silicon rubber or the like), and the circuit configuring the circuit layer 400 is manufactured in a method of printing a conductive ink or etching an electrically conductive material on the circuit plate 405.

The control chip 500 is attached to the circuit layer 400 through wire bonding, flip chip bonding or the like.

The cover 600 can be manufactured using a flexible material or a hardening material such as plastic to stabilize mechanical and electrical bonding between the circuit layer 400 and the wire layer 300 formed under the circuit layer 400.

As shown in FIG. 9, stacking of the electrode layer 200 and the wire layer 300 is accomplished by forming a via (plating) by applying a conductive material 350 on the back side of the electrode 235 of the electrode layer 200 and injecting a conductive material 350 such as a conductive ink, a conductive adhesive or the like into the connection hole 330 formed on the wire layer 300 so that the electrode 235 and the wire 335 are electrically connected to each other.

As shown in FIG. 10, stacking of the wire layer 300 and the circuit layer 400 is accomplished by inserting the electrode bar 370 inserted in the connection hole 330 formed near the center portion of the wire layer 300 and protruded upward into the electrode connection unit 410 of the circuit layer 400 to penetrate the circuit layer 400 so that the wire layer 300 and the circuit layer 400 are electrically connected to each other.

As shown in FIG. 10, stacking of the circuit layer 400 and the cover 600 is accomplished by forming an insertion hole 650 concave upward on the bottom of the cover 600 and forcibly inserting the electrode bar 370 penetrating the circuit layer 400 into the insertion hole 650 so that the circuit layer 400 and the cover 600 are connected to each other.

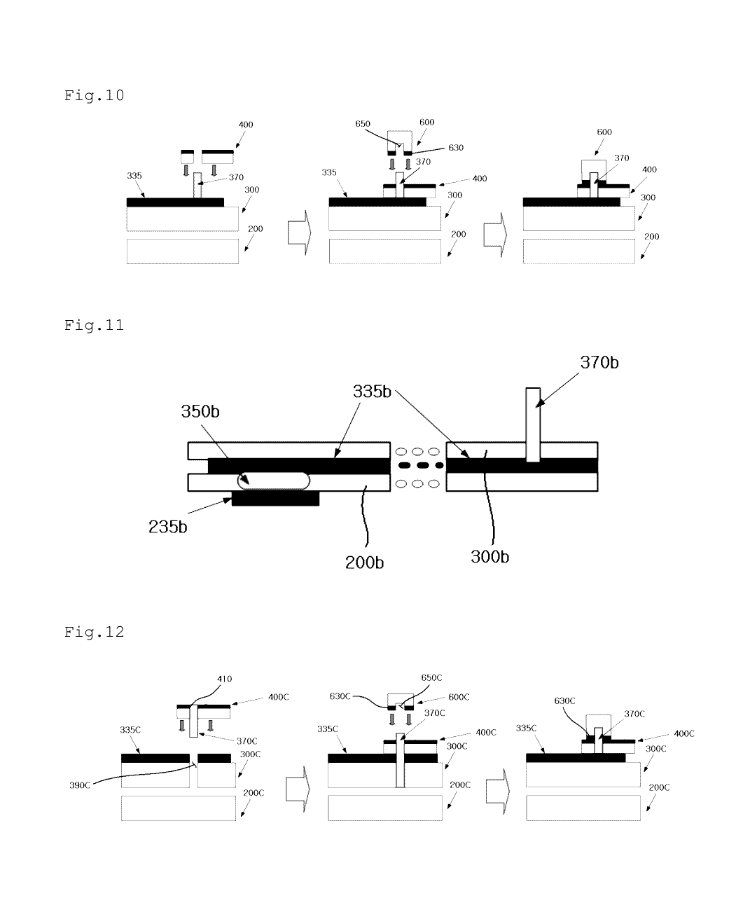

As shown in FIG. 11, stacking of the electrode layer 200b and the wire layer 300b is accomplished by applying a conductive material 350b between the electrode 235b of the electrode layer 200b and the wire 335b of the wire layer 300b so that the electrode layer 200b and the wire layer 300b are electrically connected to each other, and an electrode bar 370b can be provided to penetrate the wire layer 300b and protrude upward while the bottom of the electrode bar 370b contacts with the wire 335b.

As shown in FIG. 12, stacking of the wire layer 300c and the circuit layer 400c is accomplished by inserting the electrode bar 370c inserted in the electrode connection unit 410c of the circuit layer 400c and protruded downward into the connection hole 390c of the wire layer 300c so that the wire layer 300c and the circuit layer 400c are electrically connected to each other.

In addition, stacking of the circuit layer 400c and the cover 600c is accomplished by forming an insertion hole 650c concave upward on the bottom of the cover 600c and forcibly inserting the electrode bar 370c inserted into the circuit layer 400c and protruded upward into the insertion hole 650c so that the circuit layer 400c and the cover 600c are connected to each other.

If the photographing patch 100 is formed as described above, electrical impedance can be measured by contacting the photographing patch 100 onto the skin of a user.

In the present invention, diagnosis of breast cancer is described as an example, and since the photographing patch 100 is formed of a flexible material, it can be bent in the form of a brassiere to correspond to the shape of breasts as shown in FIG. 2.

Accordingly, the electrical impedance tomography device according to an embodiment of the present invention is a structure of providing a pair of photographing patches 100 of a brassiere shape and connecting an external device 900 having an application installed therein to the pair of photographing patches 100 through a communication cable C.

First, the surface of breasts to be photographed is cleanly wiped using a wet tissue or the like, and a separate oil or the like is lightly applied on the breasts.

Then, as shown in FIG. 1, the photographing patches 100 of a brassiere shape are tightly attached to the surface of the breasts so that all the electrodes 235 of the electrode layer 200 may electrically contact with the surface of the breasts.

In the present invention, ninety or sixty four electrodes 235 can be evenly arranged on the surface of the photographing patches 100 along the curved surface of the breasts.

Accordingly, since the surface of the breasts is divided into a plurality of areas and a predetermined number of electrodes 235 are placed in each of the areas and attached to the surface of the breasts, the electrodes 235 are three-dimensionally and evenly distributed on the surface of the breasts.

Then, the communication cable C connected to the photographing patches 100 is connected to the external device 900 such as a smart phone, a tablet computer, a notebook computer or the like, and a separate application program installed in the external device 900 is executed.

Through the application program, it can be confirmed whether or not an image can be taken by measuring contact impedance between all the electrodes 235 and the surface of the breasts, and impedance tomography is performed on the breasts after confirming whether or not all the electrodes 235 contact with the skin of the user by displaying whether or not an image can be taken on the external device 900.

The external device 900 activates the current generation unit 530 by sending a control signal to the control unit 510 of the control chip 500 and sets a frequency, an amplitude and the like of the current, i.e., an AC signal.

Next, the electrodes 235 provided on the electrode layer 200 are selected in pairs through the switching unit 520, and a certain current generated through the current generation unit 530 is applied to any one pair of electrodes 235.

The voltage sensor unit 540 measures voltage between the electrodes 235 of the pairs other than the pair of electrodes 235 to which the current is applied, and the conversion unit converts a measured analog data into a digital data, and the converted digital data is temporarily stored in the buffer unit 560.

The digital data stored in the buffer unit 560 is wiredly or wirelessly transmitted to the external device 900 through the communication unit 570.

The external device 900 receives the electrical signal measured through the photographing patch 100 and measures impedance at a ratio of the current applied through the current generation unit 530 to the voltage measured through the voltage sensor unit 540.

Next, an impedance matrix of all impedance values can be obtained by repeatedly performing this process on the electrodes 235 of the other pairs.

If the impedance matrix is calculated through the measured impedance values, the external device 900 calculates distribution of impedance inside the skin by tracing back the internal structure of the skin using the impedance matrix and displays images of different distance in the depth direction and distribution of relative impedance values in each image, as well as the image obtained through the tomography.

The impedance tomographic image according to the depth of the skin is restored and displayed as a three-dimensional tomographic image using the images and the distribution of impedance.

Generally, since impedance of a cancer cell is only one third of the impedance of a normal cell, a part of low impedance, i.e., a part suspected as a cancer lesion, can be identified through this image.

The electrical impedance tomography device according to the present invention as described above may stably measure impedance of any curved part of a body by forming a large number of electrodes on a substrate of a flexible material.

In addition, the electrical impedance tomography device is extremely small and light since it can be manufactured in the form of a brassiere or the like by integrating a semiconductor chip on a substrate close to the electrodes, and a tomographic image can be taken through simple handling such as pressing a photographing button provided in the application, like taking a picture using a smart phone, by using a smart phone, a tablet PC or the like as an external device.

Accordingly, a disease such as breast cancer or the like can be conveniently and correctly self-diagnosed in general homes.

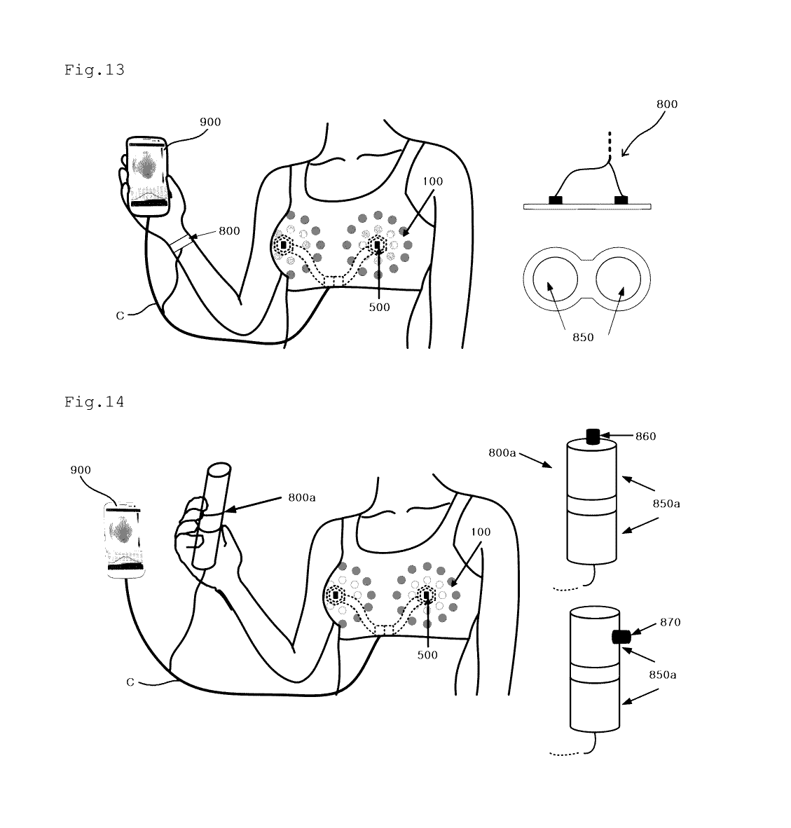

FIGS. 13 to 15 are views showing different cases of using an electrical impedance tomography device according to an embodiment of the present invention.

In a use case of another form using the electrical impedance tomography device according to the present invention, impedance values are obtained at a ratio of current to voltage by attaching a current reference electrode and a voltage reference electrode configuring a reference patch to another part of a body apart from a part of the body (breasts) for measuring impedance, flowing the current between one of the electrodes attached to the part of the body to be measured and the current reference electrode, and measuring the voltage between other electrodes attached to the breasts and the voltage reference electrode.

First, as shown in FIG. 13, a reference patch 800 formed with two reference electrodes can be used by attaching the reference patch to a wrist.

A pair of reference electrodes 850 are provided in the reference patch 800, and a wire is attached to each of the reference electrodes 850 and connected to the circuit layer of the photographing patch 100 together with the communication cable C.

At this point, a photographing button for performing photographing can be provided on the reference patch 800.

As shown in FIG. 14, a reference patch 800a formed with two reference electrodes can be provided in the shape of a bar that can be held with a hand.

A pair of reference electrodes 850a formed of thin metal film are arranged up and down on the surface of the reference patch 800a, and when the electrical impedance tomography device is used, a user holds and wraps the reference patch 800a with a palm and tightly attaches both of the upper and lower reference electrodes 850a to the palm.

A wire is attached to each of the reference electrodes 850a and connected to the circuit layer of the photographing patch 100 together with the communication cable C.

The photographing buttons 860 and 870 for performing photographing can be provided in the upper portion or on the side surface of the reference patch 800.

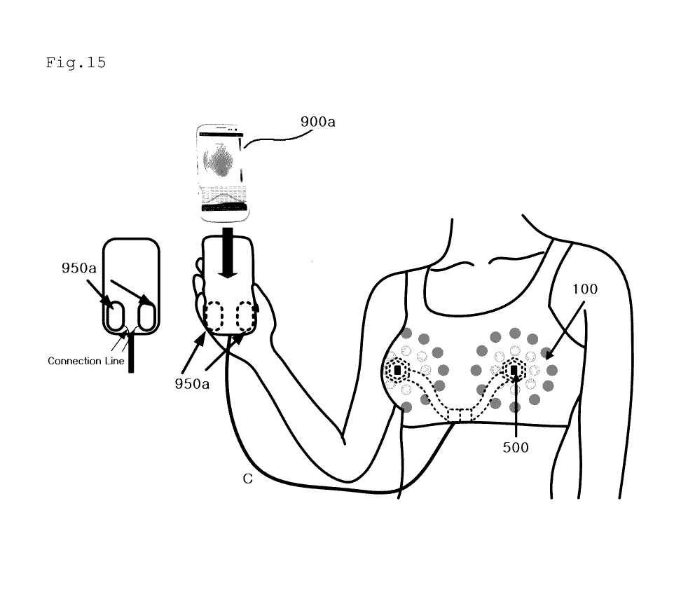

As shown in FIG. 15, the electrical impedance tomography device can be provided such that two reference electrodes 950a are formed on the back side of an external device 900a and, when a user holds the external device 900a with a hand, a finger or a palm can be touched to the reference electrodes 950a.

As a method of forming the reference electrodes 950a on the back side of the external device 900a, when the external device 900a is manufactured, the reference electrodes 950a can be implemented on the outer surface of the external device 900a to be internally connected to the external device 900a, or the reference electrodes 950a can be installed on a separate cover of the external device 900a.

Each of the reference electrodes 950a is connected to the circuit layer of the photographing patch 100 together with the communication cable C.

Impedance is measured at a ratio of current to voltage by applying the current between any one of the pair of reference electrodes configuring the reference patch described above and any one of the electrodes provided in the photographing patch 100 and measuring the voltage between the other reference electrode and the other electrodes 235 provided in the photographing patch 100.

The impedance matrix can be obtained by repeating this process for all the other electrodes.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

The electrical impedance tomography device according to the present invention can stably connect a large number of electrodes without reluctance by directly attaching small, light and soft electrodes on the surface of the skin.

In addition, the electrical impedance tomography device can be manufactured in the form of a general brassiere by integrating all electronic functions in a semiconductor chip, and since tomographic images can be taken and data can be collected, image-processed and analyzed by simple handling of the device through an application of a smart phone or a tablet computer, breast cancer can be conveniently and correctly self-diagnosed in an early stage in general homes.

1. An electrical impedance photographing patch comprising a plurality of electrodes contacting with skin, applied with an electrical signal and arranged on a flexible substrate, wherein the photographing patch measures impedance, i.e., an electrical signal, of the skin of a measurement target placed between the plurality of electrodes and transmits the electrical signal to an external device which restores the electrical signal into a three-dimensional tomographic image and displays the tomographic image.

2. The patch according to claim 1, wherein an electrode plate, a wire plate and a circuit plate configuring the substrate is formed of any one or more of a paper product, a natural fiber, a synthetic fiber, non-woven cloth, polymer, plastic, pulp, paper and silicon rubber.

3. The patch according to claim 1, wherein a cover configuring the substrate is formed of a flexible material or plastic.

4. The patch according to claim 1, wherein the electrodes, wires and circuits configuring the substrate are formed by printing an ink containing a conductive material, adhering cloth, non-woven cloth, paper or a flexible substrate having conductivity, infiltrating a conductive organic matter, configuring a metallic sticker or adhering a small metal plate.

5. The patch according to claim 1, wherein the photographing patch is a flexible brassiere shape targeting breasts of a woman as a measurement target.

6. An electrical impedance tomography device comprising:
a photographing patch including a plurality of electrodes contacting with skin, applied with an electrical signal and arranged on a flexible substrate, the photographing patch for measuring an electrical signal output through the skin of a measurement target placed between the plurality of electrodes; and
an external device for receiving the electrical signal measured through the photographing patch, restoring electrical impedance into a three-dimensional tomographic image, and displaying the tomographic image.

7. The device according to claim 6, wherein an electrode plate, a wire plate and a circuit plate configuring the substrate is formed of any one or more of a paper product, a natural fiber, a synthetic fiber, non-woven cloth, polymer, plastic, pulp, paper and silicon rubber.

8. The device according to claim 6, wherein a cover configuring the substrate is formed of a flexible material or plastic.

9. The device according to claim 6, wherein circuits configuring electrode, wire and substrate layers are formed by printing an ink containing a conductive material, adhering cloth, non-woven cloth, paper or a flexible substrate having conductivity, infiltrating a conductive organic matter, configuring a metallic sticker or adhering a small metal plate.

10. The device according to claim 6, wherein the plurality of electrodes arranged on the substrate is arranged in a shape of a regular pattern to evenly contact throughout the skin of the management target.

11. The device according to claim 10, wherein the shape of a regular pattern of the plurality of electrodes is a radial or matrix shape.

12. The device according to claim 6, wherein the photographing patch includes a control chip implemented in a semiconductor circuit as a module of one piece, and the control chip controls the device to select some of the plurality of electrodes to supply current and select other electrodes to measure voltage, converts an analog signal measured by the plurality of electrodes into a digital signal, and transmits the digital signal to the external device.

13. The device according to claim 6, wherein the photographing patch includes:
an electrode layer provided with the plurality of electrodes;
a wire layer stacked on a top of the electrode layer and provided with a wire electrically connected to the electrodes; and
a circuit layer stacked on a top of the wire layer and provided with an electrode connection unit electrically connected to the wire.

14. The device according to claim 13, wherein the electrode layer includes:
an electrode plate of a plate shape; and
an electrode terminal configured of a plurality of electrodes and provided in a radial shape from a center of the electrode plate.

15. The device according to claim 14, wherein the electrode terminal is configured of a predetermined number of electrodes and configured in plurality to be equally spaced in a circumferential direction.

16. The device according to claim 13, wherein the wire layer includes:
a wire plate of a plate shape;
a connection hole formed to penetrate the wire plate at a position corresponding to the electrode; and
a wire formed to be extended from the connection hole toward a center of the wire plate.

17. The device according to claim 13, wherein the circuit layer includes:
a circuit plate of a plate shape;
an electrode connection unit electrically connected to the wire and provided in plurality on the circuit plate to be equally spaced in a circumferential direction; and
a control chip for supplying an electrical signal to the electrode, receiving an electrical signal output from the electrode and transferring the electrical signal to the external device.

18. The device according to claim 12, wherein the control chip includes:
a switching unit for turning on or off electrical connection to the electrode;
a current generation unit for generating an electrical signal applied to the electrode;
a voltage sensor unit for receiving an electrical signal output from the electrode;
a communication unit for transmitting the electrical signal input through the voltage sensor unit to the external device; and
a control unit for controlling the switching unit to select an electrode for receiving the electrical signal generated through the current generation unit and electrically connect electrodes other than the electrode receiving the electrical signal to the voltage sensor unit.

19. The device according to claim 13, wherein a communication connection unit connected to the communication cable is provided in the circuit layer.

20. The device according to claim 13, wherein a cover stacked to cover the electrode connection unit is provided on a top of the circuit layer.

21. The device according to claim 13, wherein the electrode layer and the wire layer are electrically connected to each other by inserting a conductive material in a connection hole formed in the wire layer; the wire layer and the circuit layer are electrically connected to each other by inserting an electrode bar inserted in the connection hole formed near a center portion of the wire layer and protruded upward into the electrode connection unit of the circuit layer to penetrate the circuit layer; and the circuit layer and a cover are connected to each other by forming an insertion hole concave upward on a bottom of the cover and forcibly inserting the electrode bar penetrating the circuit layer into the insertion hole.

22. The device according to claim 13, wherein the wire layer and the circuit layer are electrically connected to each other by inserting an electrode bar inserted in the electrode connection unit of the circuit layer and protruded downward into a connection hole of the wire layer.

23. The device according to claim 6, wherein impedance is measured at a ratio of current to voltage by grouping the plurality of electrodes in pairs of two, applying the current to a certain pair of electrodes, and measuring the voltage between the electrodes of the other electrode pairs.

24. The device according to claim 6, further comprising a reference patch configured to include a reference electrode contacting with skin of a position spaced apart from the photographing patch to measure impedance between the photographing patch attached at a position of the measurement target and the reference patch.

25. The device according to claim 24, wherein the reference patch is attached to a cover of the external device or a bar shape object that can be held with a palm.

26. The device according to claim 24, wherein the reference patch further includes a photographing button.

27. The device according to claim 24, wherein the reference electrode is a pair, and impedance is measured at a ratio of current to voltage by applying the current between any one of the reference electrodes and any one of the electrodes provided in the photographing patch and measuring the voltage between the other reference electrode and the other electrodes provided in the photographing patch.

28. The device according to claim 6, wherein after calculating an impedance matrix, the external device calculates distribution of impedance inside the skin by tracing back an internal structure of the skin using the impedance matrix and three-dimensionally displays an impedance tomographic image according to depth of the skin using the distribution of impedance.

29. The device according to claim 6, wherein the external device directs a control chip embedded in the photographing patch to control the photographing patch to measure surface contact impedance of a part of a body to be measured, determines whether or not an image can be taken based on the surface contact impedance, and displays the image.

30. The device according to claim 6, wherein since an interface for wired or wireless communication is formed in the circuit layer configuring the photographing patch, a measured electrical signal can be transmitted to the external device and displayed thereon, and a control signal can be received from the external device.

31. The device according to claim 30, wherein the photographing patch is supplied with power from the external device in a case of a wired communication.

32. The device according to claim 30, wherein the photographing patch is supplied with power by attaching a small battery to the circuit layer in a case of a wireless communication.
